(19) United States
(12) Patent Application Publication
(10) Pub. No.: US 2006/0004318 A1
(43) Pub. Date: Jan. 5, 2006

(54) Disposable device that supplies a material to a vagina

(76) Inventors: Ann Marie Przepasniak, Appleton, WI (US); Amanda O'Connor, Appleton, WI (US); MaryAnn Zunker, Oshkosh, WI (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/881,025

(22) Filed: Jun. 30, 2004

(51) Int. Cl. A61F 13/20

(52) U.S. Cl. 604/14; 604/15

(57) ABSTRACT

The invention relates to a disposable device for supplying a material to a vagina. The disposable device includes a housing, a membrane and a material (e.g., a liquid) that is enclosed by the membrane. The disposable device further includes a core that is positioned within the housing. The core is movable within the housing to open the membrane. In some embodiments, the core includes a handle and a swab secured to the handle such that the swab absorbs the liquid that was originally within the membrane. In some embodiments, the membrane includes an elastic member that seals the liquid inside the membrane and the housing includes resilient members that close the second end of the housing. The resilient members engage the elastic member such that the elastic member opens as the core moves within the housing to force the resilient members apart and dispense the material from the membrane.

### FIELD OF THE INVENTION

Some embodiments of the present invention relate to a disposable vaginal device, and in particular to a disposable device that supplies a material to a vagina.

### BACKGROUND OF THE INVENTION

A typical vagina is a relatively long and tube-like structure that extends from the cervical os (i.e., entry to the womb) down to the introitus. A vagina will normally emit secretions from glands in the vaginal canal and the interior of the uterus. As an example, mucous can develop during the phase of the menstrual cycle where the endometrium thickens and glands in the endometrium secrete mucous.

Vaginal secretions are typically not harmful but occasionally debris in the form of blood or seminal fluid can accumulate within the vagina. When debris accumulates within the vagina it may be beneficial to be able to instill the vagina with fragrances, cleansers, medications, germicides and/or deodorants (among other items).

One method of instilling an agent into a vagina includes using a product to force a fluid up into the vagina. Some of these types of products include a nozzle that is attached to a bottle filled with a solution (e.g., a cleansing solution). The solution is forced through the nozzle into the vagina under pressure.

One drawback with such products is that bacteria can be forced into, or past, the cervical os when too much pressure is applied. In addition, these types of products can rinse out healthy, normal flora (lactobacillus) along with unwanted materials, such as semen, discharges and bacteria.

There are some vaginal products that are applied to the vagina as films, suppositories, creams and gels (among others) which are later discharged from the body. One drawback with such products is that they are difficult to insert up into the vagina. Another drawback is that such items are discharged from the vagina over time such that a pantiliner is often required for a period of time after the items are applied to the vagina to prevent staining in undergarments.

There are some vaginal products that are designed to mechanically clean a vagina without using pressurized fluid. These types of devices are typically inserted into the vagina and mechanically manipulated in order in clean the vagina. One drawback with using such devices is that they are sometimes difficult to properly insert into the vagina.

There is a need for a vaginal device that is able to apply a material to the vagina without causing the vagina to become dry and irritated. The vaginal device should also be able to deliver materials into the vagina with minimal mess and without using high pressure fluid. It would also be desirable if the device was small, portable, discreet and sanitary while being able to store materials that get applied to the vagina for an extended period of time.

### SUMMARY OF THE INVENTION

One aspect of the invention relates to a disposable device for supplying a material to a vagina. The disposable device includes a housing, a membrane and a material (e.g., a liquid) that is at least partially enclosed by the membrane. The device further includes a core that is positioned within the housing. The core is movable within the housing to open the membrane.

In some embodiments, the housing includes a first end and a second end and the core includes a handle and a swab that is secured to the handle. The membrane may be ruptured such that the swab absorbs the liquid. There are some embodiments where the membrane may be positioned between the swab and the second end of the housing before the membrane is ruptured and other embodiments where the membrane may be positioned between the swab and the first end of the housing before the membrane is ruptured.

In some embodiments, the core may be movable within the housing between a first position where the swab is at least partially within the housing and a second position where the swab is extended through a second end of the housing. The membrane may be ruptured when the core is at any location between the first position and the second position as long as the swab is still at least partially within the housing.

In some embodiments, the housing includes resilient members that close the second end of the housing. In addition, the membrane may include an elastic member that seals the liquid inside the membrane. The tips of the resilient members engage the elastic member of the membrane such that the elastic member opens as the core moves within the housing to force the resilient members apart. The core may continually be moved relative to the housing until the core forces the material through the elastic band to dispense the material from the membrane to a vagina.

Another aspect of the invention relates to a method of supplying a material to a vagina. The method includes positioning a disposable device near a vagina. As an example, the disposable device may be inserted into the vagina. The disposable device includes a housing and a core that is positioned within the housing. The disposable device further includes a membrane and a material (e.g., a liquid) that is at least partially enclosed by the membrane. The method further includes moving the core relative to the housing to open the membrane.

In some embodiments, moving the core relative to the housing to open the membrane includes rupturing the membrane. It should be noted that moving the core relative to the housing may include (i) moving the core to engage the core with the membrane; or (ii) moving the core to engage the membrane with the housing.

In some embodiments, the core includes a handle and a swab that is secured to the handle such that rupturing the membrane may include rupturing the membrane with the swab. In other embodiments, rupturing the membrane may include rupturing the membrane with the handle.

In some embodiments, the housing includes a first end and a second end and the membrane includes an elastic member that seals the material inside the membrane. The second end of the housing includes resilient members that close the second end of the housing. The tips of the resilient members engage the elastic member of the membrane such that moving the core includes forcing apart the resilient members to open the elastic member. The method may further include engaging a stop on the core with the housing such that the core is prevented from further movement into the housing. The stop may be positioned on the core such that the stop prevents the core from moving far enough into housing to dislodge the membrane from the housing.

### BRIEF DESCRIPTION OF THE DRAWINGS

The above-mentioned and other features will become more apparent and better understood by reference to the following description of the invention taken in conjunction with the accompanying drawings.

FIG. 1 is a side view illustrating an example disposable device for supplying a material to a vagina.

FIG. 2 is a section view of the disposable device shown in FIG. 1 taken along line 2-2 in FIG. 1.

FIG. 3 is a section view illustrating the disposable device shown in FIG. 2 as a membrane within the device is being ruptured.

FIG. 4 is a section view similar to FIG. 2 illustrating another example embodiment of the disposable device shown in FIG. 1.

FIG. 5 is a section view illustrating the disposable device shown in FIG. 4 as a membrane within the device is being ruptured.

FIG. 6 is a section view illustrating another example disposable device with a core of the device in a first position.

FIG. 7 is a section view of the disposable device shown in FIG. 6 with a core of the device in a second position.

FIG. 8 is a section view of the disposable device shown in FIGS. 6 and 7 as a membrane within the device is being ruptured.

FIG. 9 is a section view illustrating another example embodiment of the disposable device shown in FIG. 6 with a core of the device in a first position.

FIG. 10 is a section view of the disposable device shown in FIG. 9 with a core of the device in a second position.

FIG. 11 is a section view of the disposable device shown in FIGS. 9 and 10 as a membrane within the device is being ruptured.

FIG. 12 is a section view illustrating another example disposable device for supplying a material to a vagina.

FIG. 13 is a section view of the disposable device shown in FIG. 12 as a core of the device moves within a housing.

FIG. 14 is a section view of the disposable device shown in FIGS. 12 and 13 as the core of the device forces a material through an elastic band of a membrane.

FIG. 15 is a section view of the disposable device shown in FIGS. 12-14 as the core of the device forces the membrane through the elastic band.

FIG. 16 is a section view illustrating another example disposable device for supplying a material to a vagina.

FIG. 17 is a section view of the disposable device shown in FIG. 17 as a core of the device ruptures a membrane within a swab.

FIG. 18 is a section view of the disposable device shown in FIGS. 16 and 17 after a material within the membrane is absorbed into the swab.

FIGS. 19-21 are schematic views illustrating an example method of supplying a material to a vagina.

FIGS. 22-24 are schematic views illustrating another example method of supplying a material to a vagina.

FIGS. 25-27 are schematic views illustrating still another example method of supplying a material to a vagina.

### DESCRIPTION OF THE INVENTION

The disposable device of the present invention may provide an efficient, healthy and low-cost way to supply a material to a vagina. The disposable device may also provide an effective way to clean a vagina.

As used herein, “disposable device” includes a device that is disposed of after use and is not intended for reuse. The disposable device described herein may be especially useful to an individual that needs to clean and/or apply a material to their vagina in a variety of situations.

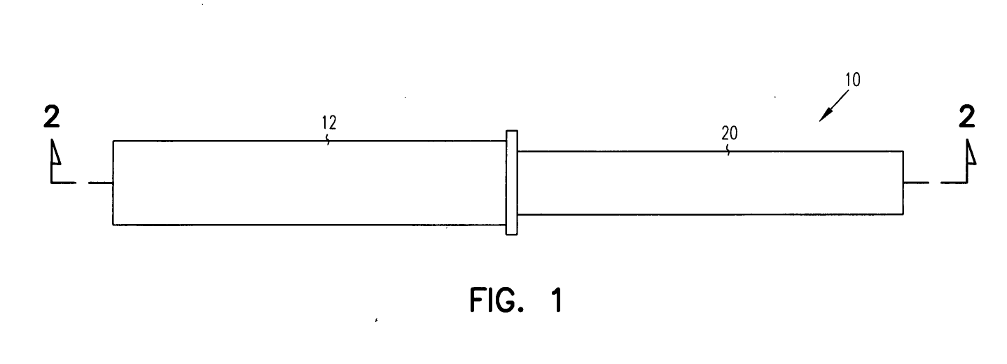

FIGS. 1-5 illustrate an example disposable device 10 for supplying a material to a vagina. The disposable device 10 includes a housing 12, a membrane 14 and a material (e.g., liquid 16) that is at least partially enclosed by the membrane 14. The device 10 further includes a core 20 that is positioned within the housing 12. The core 20 is movable within the housing 12 to open the membrane 14. In some example embodiments, the membrane 14 is ruptured by the housing 12 and/or the core 20.

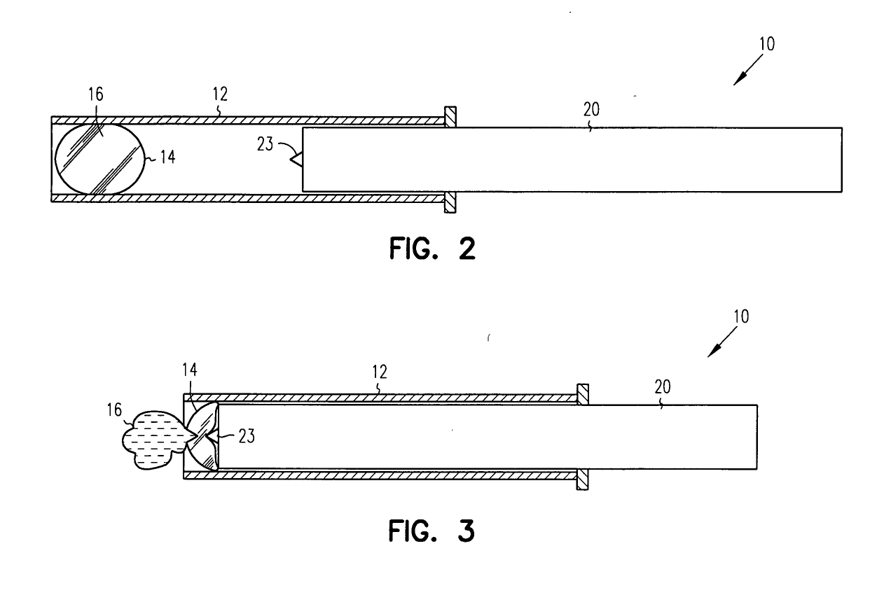

FIG. 2 shows an example embodiment where the membrane 14 is attached to the housing 12 before being ruptured while FIG. 3 shows the membrane 14 being ruptured. In the example embodiment illustrated in FIG. 2, the membrane 14 and the housing 12 enclose the liquid 16 before the membrane 14 is ruptured to dispense the liquid 16. It should be noted that in some embodiments the liquid 16 is enclosed only by the membrane 14.

As shown in FIGS. 2 and 3, the core 20 may include a poke 23 that ruptures the membrane 14 as the core 20 engages the membrane 14. The poke 23 may have any configuration that facilitates rupturing the membrane 14. In addition, the core 20 may include a plurality of pokes such that multiple pokes are used to rupture membrane 14.

The membrane 14 may be made from a material that is non-toxic, low cost and hygienic. Some example materials that may be used for the membrane 14 include plastic, amorphous polyalphaolefin, propylene-ethylene co-polymer, ethylene-butene polymer, polyethylene oxide with polyesters and/or polyurethanes, polyethylene oxide with ethylene co-polymer, PVA with ethylene co-polymer, methylmethacrylate with PVA and latex (among other materials).

In some embodiments, the membrane 14 may be a combination of polymers. As an example, some combinations of polymers may include multiple layers with one of the layers being water soluble and another of the layers being water insoluble. It should be noted that embodiments are contemplated where one polymer is a coating while another polymer is a film.

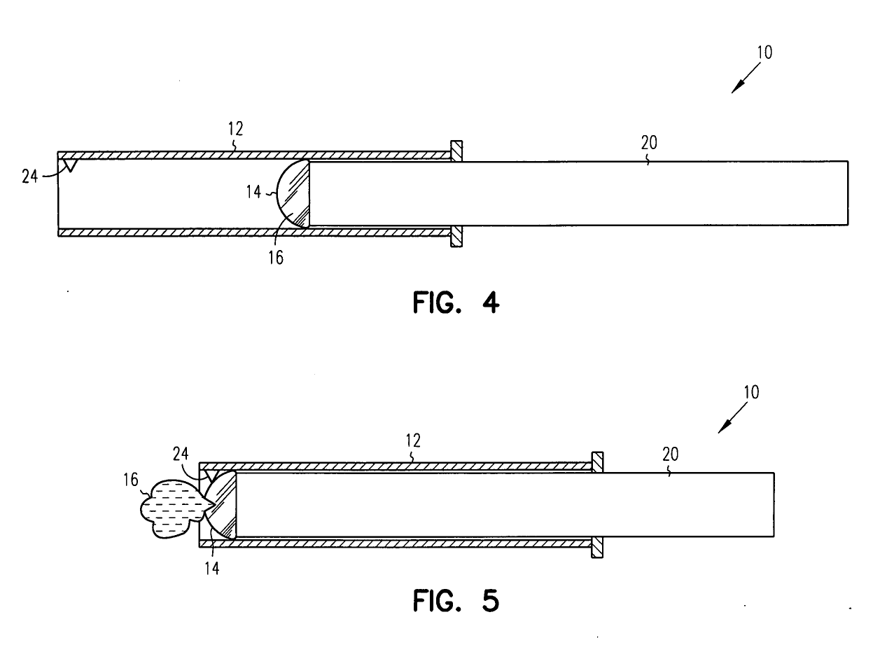

FIG. 4 shows an example embodiment where the membrane 14 is attached to the core 20 before being ruptured while FIG. 5 shows the membrane 14 being ruptured. In the example embodiment illustrated in FIG. 4, the membrane 14 and the core 20 enclose the liquid 16 before the membrane 14 is ruptured to dispense the liquid 16. It should be noted that in some embodiments the liquid 16 is enclosed only by the membrane 14.

As shown in FIGS. 4 and 5, the housing 12 may include a poke 24 that ruptures the membrane 14 as the membrane 14 engages the housing 12. The poke 24 may have any configuration that facilitates rupturing the membrane 14. In addition, the housing 12 may include a plurality of pokes such that multiple pokes are used to rupture membrane 14.

In the example embodiments illustrated in FIGS. 1-5, the housing 12 is a cylindrical tube. It should be noted that the cross-section of the housing 12 may take a variety of forms (e.g., circle, square, rectangle and polygon) depending on the properties that are desired within the disposable device 10.

The housing 12 may be made from a material that is non-toxic, low cost and hygienic. Some example materials that may be used for housing 12 include polyethylene and polypropylene (among other materials). In some embodiments, the housing 12 may be made from a combination of materials.

The width (i.e., outer diameter) of the housing 12 may vary depending on the functional properties that are desired of the disposable device 10. In some embodiments, the housing may be small enough to make it easy for a user to insert the disposable device 10 into the vagina while being wide enough to give the housing 12 sufficient space to store the core 20. As an example, the width of the housing 12 may be between 25 mm and 60 mm.

The wall thickness of the housing 12 should be large enough to provide sufficient rigidity to the disposable device 10 during vaginal insertion. As an example the wall thickness may be between 2 mm and 10 mm. In addition, the length of the housing 12 may be between 60 mm and 150 mm. The relative lengths of the housing 12 and the core 20 may vary depending on the desired properties of the disposable device 10.

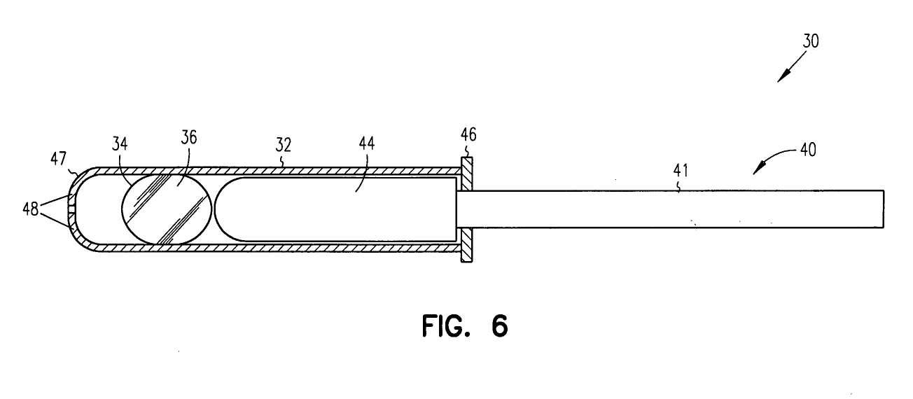

FIGS. 6-11 illustrate another example disposable device 30 for supplying a material to a vagina of the present invention. The disposable device 30 includes a housing 32, a membrane 34 and a liquid 36 that is at least partially enclosed by the membrane 34. The device 30 further includes a core 40 that is positioned within the housing 32. The core 40 is movable within the housing 32 to open the membrane 34.

The core 40 includes a handle 41 and a swab 44 that is secured to the handle 41. The membrane 34 may be ruptured by the housing 32 and/or the core 40 such that the swab 44 absorbs the liquid 36.

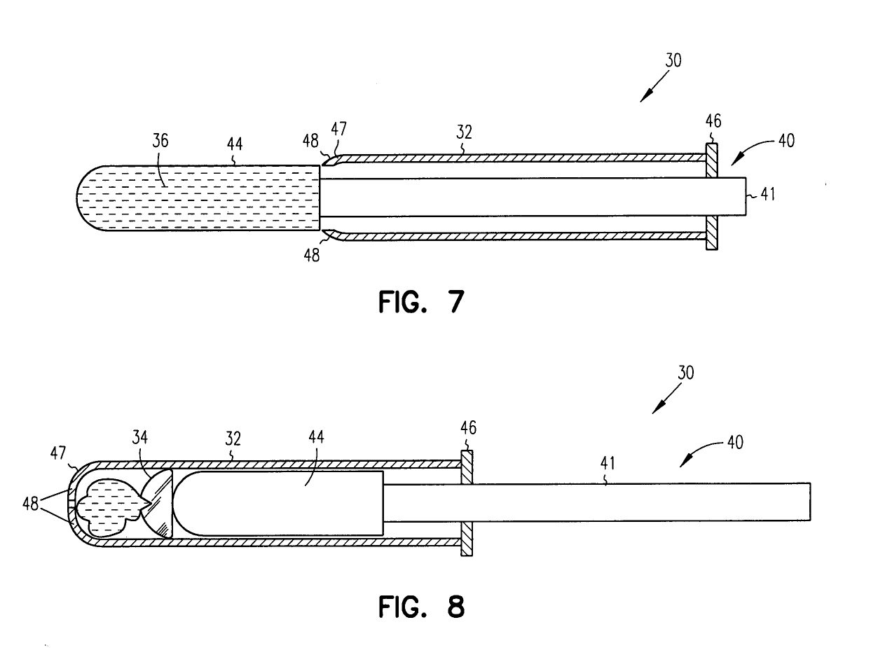

In the example embodiment illustrated in FIGS. 6-8, the housing 32 includes a first end 46 and a second end 47 such that the membrane 14 may be positioned between the swab 44 and the second end 47 of the housing 32 before the membrane 34 is ruptured. The core 40 is axially movable within the housing 32 between a first position (FIG. 6) where the swab 44 may be at least partially within the housing 32 and a second position (FIG. 7) where the swab 44 is extended through the second end 47 of the housing 32. The swab 44 may be positioned entirely within the housing 32 when the core 40 is in the first position and/or may be positioned entirely outside the housing 32 when the core 40 is in the second position.

As shown in FIG. 8, the membrane 34 may be ruptured by the swab 44. The membrane 34 may be ruptured when the core 40 is at any location between the first position and the second position as long as the swab 44 is still within the housing 32. In some embodiments, the membrane 34 may be ruptured just as the core 40 is moved from the first position.

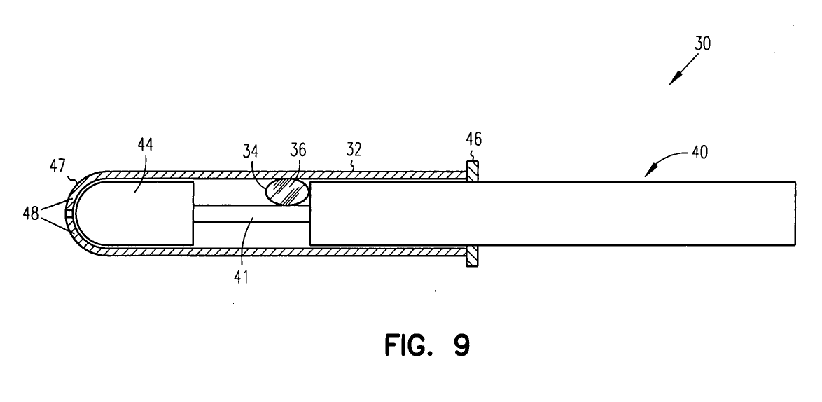

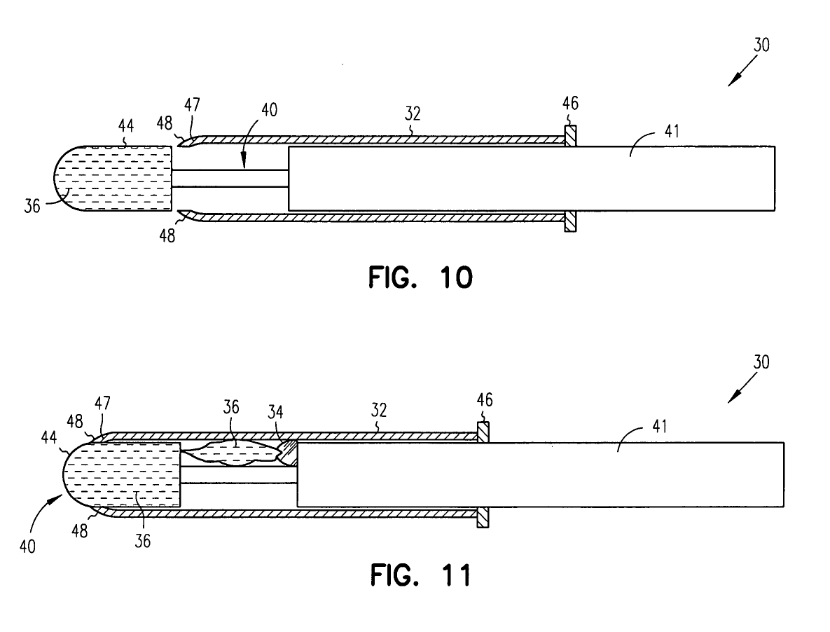

In the example embodiment illustrated in FIGS. 9-11, the membrane 34 is positioned between the swab 44 and the first end 46 of the housing 32 before the membrane 34 is ruptured. The core 40 is movable within the housing 32 between a first position (FIG. 9) where the swab 44 may be at least partially within the housing 32 and a second position (FIG. 10) where the swab 44 is extended through the second end 47 of the housing 32.

As shown in FIG. 11, the membrane 34 may also be ruptured by the handle 41. The membrane 34 may be ruptured when the core 40 is at any location between the first position and the second position as long as the swab 44 is still at least partially within the housing 32. In some embodiments, the membrane 34 may be ruptured just as the core 40 is moved from the first position.

As used herein, the term “securing” means permanently bonding. Any securing, or bonding, of the handle 41 to the swab 44 may occur at any time during the fabrication of the disposable device 30. Some of the example methods that may be used to bond the swab 44 to the handle 41 include adhesive bonding, thermal bonding and ultrasonic bonding (among others). In the illustrated example embodiment, the handle 41 is embedded in the swab 44.

In some embodiments, the swab 44 has a length that may be between 50 mm and 150 mm and a width that may be between 25 mm and 50 mm. The swab 44 may be constructed of any material that is suitable for insertion into a vagina. Some example materials include cotton, rayon, wood pulp and polymeric substances such as nonwoven fabrics, foam sponges and thermoplastics.

The swab 44 may be designed with any type of surface that enhances the disposable device's cleaning and/or adsorption properties. In addition, the surface of the swab 24 may be textured to facilitate removal of residue and film from the vagina.

In some embodiments, the swab 44 may be composed of a nonwoven fabric. As used herein, “nonwoven fabric” refers to a web or fabric (made partly or wholly of non-cellulose material) that includes individual fibers which are interlaid (randomly or in a pattern) in a knitted fabric. The nonwoven webs may be formed by many processes, including meltblowing, spunbonding and bonded carded. The nonwoven fabrics that are used in the swab 44 may be produced from polymers such as polyethylene or polypropylene.

The swab 44 may also be formed of a nonwoven fabric that is made of an array of interbonded thermoplastic fibers. The fibers may be formed from a wide variety of thermoplastic materials. Some example thermoplastic materials include polyolefins (e.g., polyethylene, polypropylene), polystyrene and polyamides (e.g., nylon). In addition, thermoplastic polymers that are elastomeric may also be used as fibers. Some example elastomeric materials include polyurethanes and block copolymers. It should be noted that blends of any of the foregoing materials may be used to form the fibers. The fibers may also include additives (e.g., wax, pigments, stabilizers and fillers) that are inserted as the fibers are fabricated to achieve one or more desired properties within the fibers.

In some embodiments, the swab 44 may include a cover material (not shown) that is placed on the swab 44 to prevent fiber sloughing and/or improve the wettablilty of the swab 44. There are also cover materials that may provide stain-masking.

As examples, the cover material may store and/or capture debris, cleansers, lubricants, spermicidal agents and medications (among other materials) before and/or during use of the disposable device 30. In addition, the swab 44 and/or cover material may be able to remove certain select materials from a vagina. Some example cover materials include spunbound, spunlace, bonded carded web and apertured film materials. In one example embodiment, the cover material is an apertured film that is formed of a polyolefin which may or may not be combined with a nonwoven fabric.

When a nonwoven fabric is used as the material for the swab 44, the basis weight of the nonwoven fabric may vary depending on the properties that are desired within the swab 44. As an example the basis weight for the nonwoven fabric may be as low as 10 grams per square meter and as high as 300 grams per square meter.

The housing 32 helps to protect the swab 44 from contamination when the core 40 is in the first position. In some embodiments, the second end 47 of the housing 32 includes resilient members 48 that at least partially close the second end 47 of the housing 32. As the core 40 is moved from the first position to the second position, the resilient members 48 are forced apart by the swab 44 (see FIGS. 7 and 10). Although the resilient members 48 are shown as relatively thin sections, the resilient members 48 may be a variety of shapes and sizes.

The handle 41 may be a variety shapes and sizes. As an example, the cross-section of the handle 41 may take a variety of forms (e.g., circle, square, rectangle and polygon) depending on the properties that are desired within the disposable device 30.

The handle 41 may be made from any material that is suitable for use in the disposable device. The handle 41 should be made from a material that is non-toxic, low cost and hygienic. Some example materials that may be used for handle 41 include paper, cardboard, polyethylene and polypropylene (among other materials). In some embodiments, the handle 41 may be made from a combination of materials.

It should be noted that handle 41 may be solid or hollow. When the handle 41 is hollow, the wall thickness of the handle 41 should be large enough to provide sufficient rigidity to the disposable device 30.

In some embodiments, the handle 41 may have a length that is between 40 mm and 250 mm. The handle 41 and the swab 44 may be sized to keep the handle 41 far enough outside the vagina when the core 40 is in the second position to allow a user to adequately manipulate the swab 44 via the handle 41. In addition, the handle 41 and the swab 44 may be sized to be short enough to allow a user to generate sufficient pressure on the vagina with the swab 44 via the handle 41. The relative lengths of the swab 44, housing 32 and handle 41 may vary depending on the desired properties of the disposable device 30.

The width (i.e., outer diameter) of the handle 41 may be small enough to make it easy for a user to insert the disposable device into the vagina while being wide enough to give the handle 41 sufficient rigidity to properly clean inside the vagina and/or apply a material to the vagina. As an example, the width of the handle 41 may be between 3 mm and 50 mm depending in part on the material that used for the handle 41.

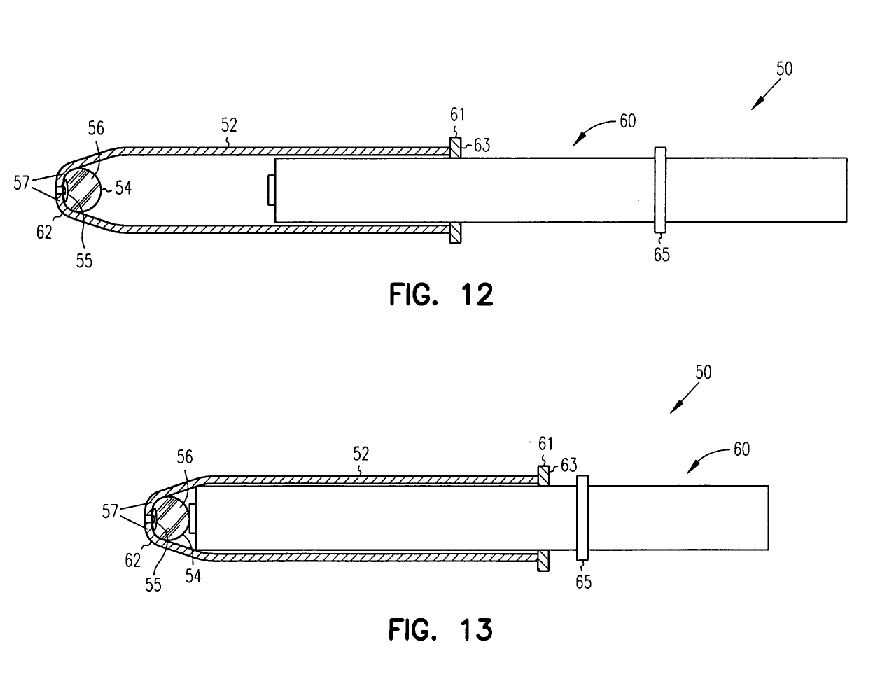

FIGS. 12-15 illustrate another example disposable device 50 for supplying a material to a vagina. The disposable device 50 includes a housing 52, a membrane 54 and a material (e.g., liquid 56) that is enclosed by the membrane 54. The device 50 further includes a core 60 that is positioned within the housing 52. The core 60 is movable within the housing 52 to open the membrane 54.

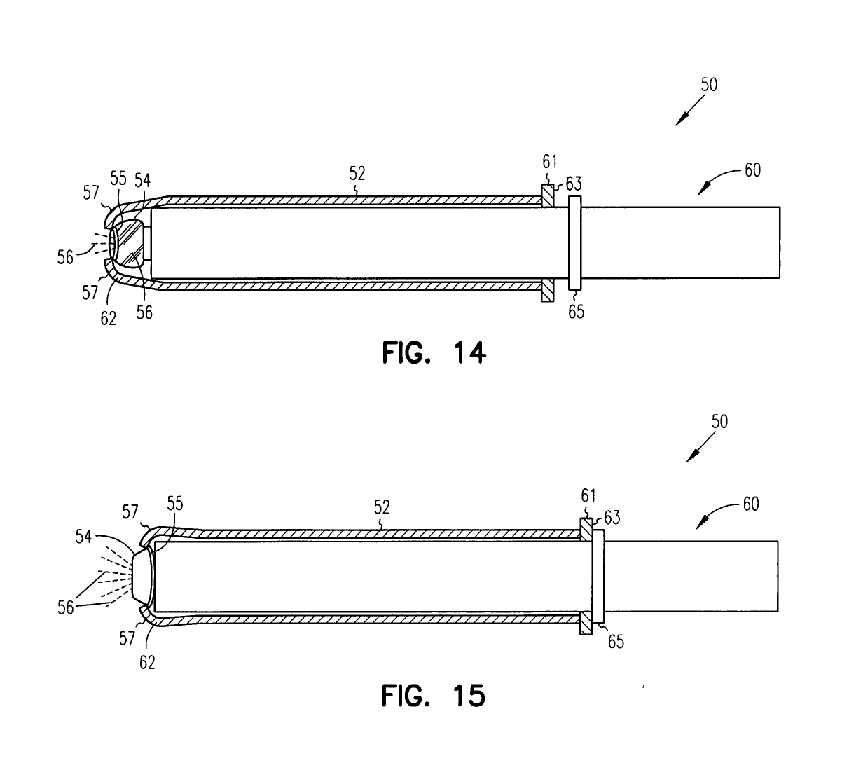

The housing 52 includes a first end 61 and a second end 62. The second end 62 of the housing includes resilient members 57 that close the second end 62 of the housing 54 (FIGS. 12 and 13).

The membrane 54 includes an elastic member 55 that seals the liquid 56 inside the membrane 54. FIG. 13 shows the position of the core 60 just as the core 60 engages the second end 62 of the housing 52. The tips of the resilient members 57 engage the elastic member 55 of the membrane 54 such that the elastic member 55 opens as the core 60 moves within the housing 52 to force the resilient members 57 apart.

The core 60 moves relative to the housing 52 until the core 60 forces the liquid 56 through the elastic band 55 (FIG. 14) to dispense the liquid 56 from the membrane 54 to the vagina (not shown in FIGS. 12-15). In some embodiments, the core 60 continues to move relative to the housing 52 such that the core 60 forces the membrane 54 through the elastic band 55 (FIG. 15) to ensure that the liquid 56 is dispensed from the membrane 54 to the vagina. The membrane 54 may need to be forced through the elastic member 55 in order to adequately dispense some materials (e.g., powders or high viscosity liquids).

The membrane 54 and the elastic member 55 may be made from a material that is non-toxic, low cost and hygienic. Some example materials that may be used for the membrane 54 and the elastic member 55 include Latex, spandex, Lycra®, Lycra® with nonwoven material, nitrile, neoprene, butadiene, ethylene-propylene, nylon, acrylic and polyester (among other materials).

In some embodiments, the membrane 54 and/or the elastic member 55 may be made from a combination of materials. In addition, the membrane 54 and the elastic member 55 may be made from the same material or different materials.

In some embodiments of the disposable device 50, the core 60 includes a stop 65. As shown most clearly in FIG. 15, the core 60 is moved relative to the housing 52 until the stop 65 on the core 60 engages the housing 52 to prevent further insertion of the core 60 into the housing 52. The stop 65 should be located on the core 60 to prevent the core 60 from dislodging the membrane 54 at the second end 62 of the housing 52. The relative lengths and widths of the core 60 and housing 52 may vary depending on the desired properties of the disposable device 50 as long as the stop 65 prevents the core 60 moving far enough into housing 52 to dislodge the membrane 54 from the housing 52.

In the example embodiment illustrated in FIGS. 12-15, the stop 65 is an annular disk that projects from the core 60 to engage the first end 61 of the housing 52. As shown most clearly in FIG. 15, the first end 61 of the housing 52 includes a projection (e.g., end cap 63) that extends into an opening in the housing 52. It should be noted that in other embodiments the housing 52 may not include an end cap 63 such that the stop 65 directly engages the wall of the housing 52 at the first end 61 of the housing 52. Many other embodiments are contemplated for the core 60, housing 52 and/or stop 65 as long as the stop 65 prevents further insertion of the core 60 into the housing 52.

The disposable devices described herein may further include a locking mechanism that precludes the membrane from being ruptured prematurely. The locking mechanism precludes premature rupturing of the membrane by preventing the core from moving relative to the housing. It should be noted that any type of locking mechanism may be incorporated into the disposable devices described herein, including locking mechanisms which are known now, or developed in the future.

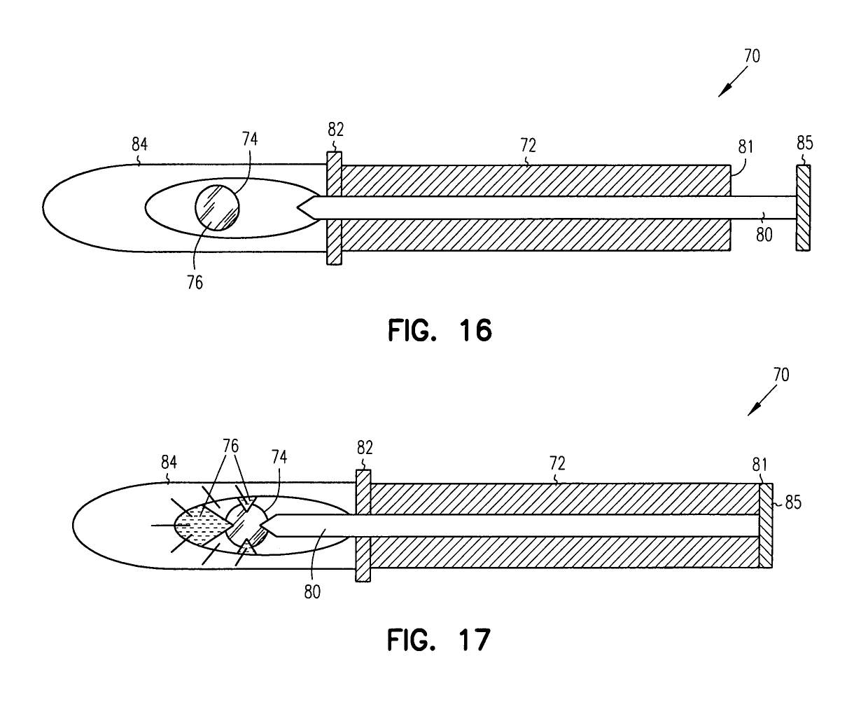

FIGS. 16-18 illustrate another example disposable device 70 for supplying a material to a vagina of the present invention. The disposable device 70 includes a housing 72 having a first end 81 and a second end 82, and a core 80 which is positioned within the housing 72. The device 70 further includes a membrane 74 and a material (e.g., liquid 76) that is enclosed by the membrane 74.

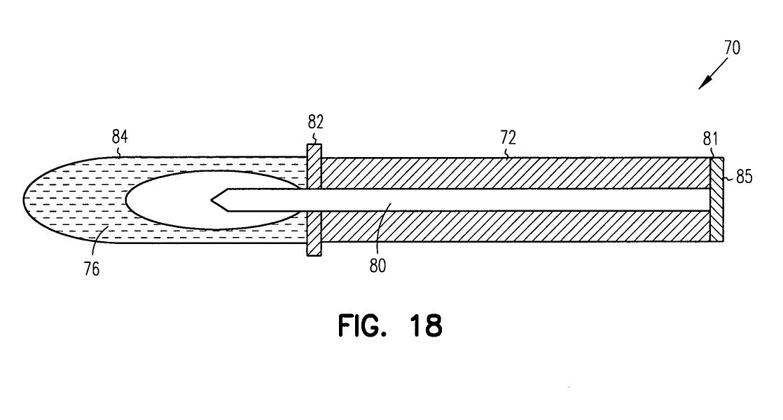

FIG. 16 shows that the membrane 74 is embedded in a swab 84 that is attached to the second end 82 of the housing 72. The core 80 is movable within the housing 72 to open (i.e., rupture) the membrane 74 within the swab 84 (see FIG. 17). As shown in FIG. 18, once the core 80 ruptures the membrane 74, the swab 84 absorbs the liquid 76.

In some embodiments of the disposable device 70, the core 80 includes a stop 85. As shown most clearly in FIG. 18, the core 80 is moved relative to the housing 72 until the stop 85 on the core 80 engages the housing 72 to prevent further insertion of the core 80 into the housing 72. The stop 85 should be located on the core 80 to prevent the core 80 from damaging the swab 84. The relative lengths and widths of the swab 84, core 80 and housing 72 may vary depending on the desired properties of the disposable device 70 as long as the stop 85 prevents the core 80 moving far enough into housing 72 to damage swab 84.

In any of the disposable devices described or reference herein, the material in the disposable device may be a cleanser. The term “cleanser” refers to any paste, gel, powder, oil, liquid or any other appropriate medium that may be used in vaginal cleansing. Some example cleansers include surfactants such as water-soluble polymers, polysorbates, glycerins, glycol-based surfactants and/or silicone-based surfactants.

The cleanser should not kill significant amounts of lactobacillus while removing unwanted debris during cleaning of the vagina. In addition, the cleanser should not contain ingredients that might lead to dry vaginal mucosal tissue.

In some embodiments, the cleanser may include a moisturizer that helps to maintain a normal hydration level within the vagina. The cleanser may also contain preservatives and other ingredients that do not disrupt the normal flora of the vaginal vault (e.g., sorbic acid, citric acid, methyl paraben and natural preservatives such as grapefruit extract).

It should be noted that the material may include other items besides cleansers such that the disposable devices described herein can be used to apply any number of materials to the vagina. Some example materials include lubricants and other inactive or active ingredients (e.g., a spermicidal agent or a medication). Another example material may be a motility enabler that facilitates sperm motility. The material may also include other items, such as water, salts, vinegars, humectants, scouring powders, thickening agents and aromas (among others).

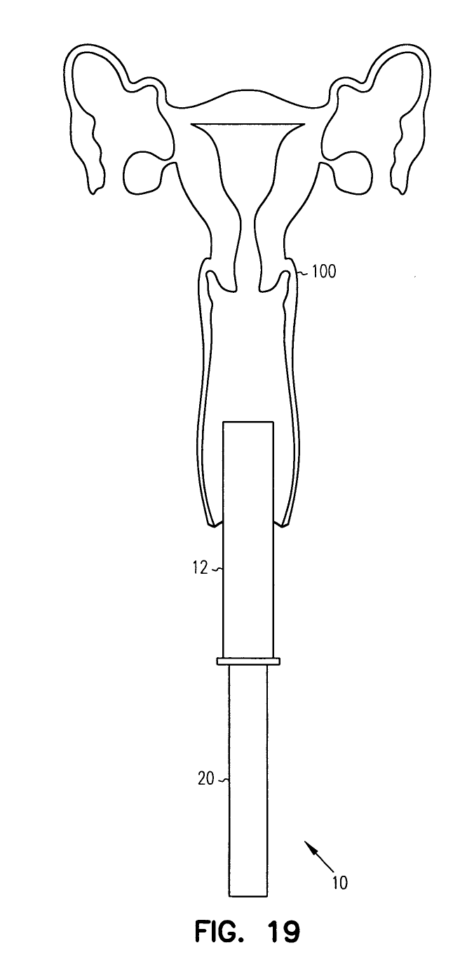

An example method of supplying a material to a vagina will now be described with reference to FIGS. 19-21. As shown in FIG. 19, the method includes positioning a disposable device 10 near a vagina 100. In the illustrated embodiments, positioning the disposable device 10 near the vagina 100 includes inserting the disposable device 10 into the vagina 100. The disposable device 10 includes a housing 12 and a core 20 that is positioned within the housing 12. The disposable device 10 further includes a membrane 14 and a material (e.g., liquid 16) that is at least partially enclosed by the membrane 14.

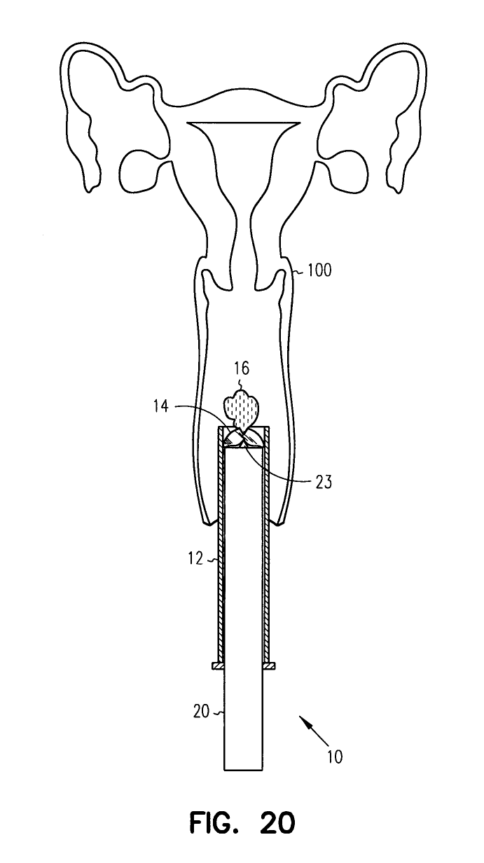

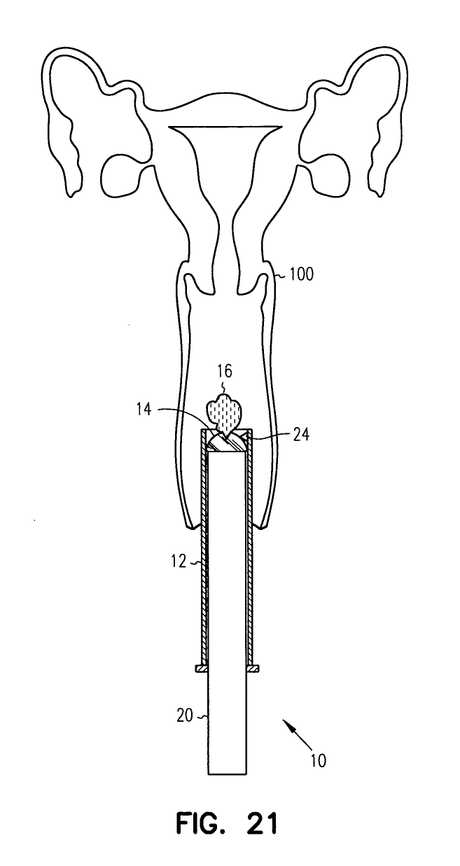

As shown in FIGS. 20 and 21, the method further includes moving the core 20 relative to the housing 12 to open the membrane 14. In some embodiments, moving the core 20 relative to the housing 12 to open the membrane 14 includes rupturing the membrane 14.

FIG. 20 shows that moving the core 20 relative to the housing 12 may include engaging the core 20 with the membrane 14 (FIG. 20). In addition, moving the core 20 may include engaging a poke 23 on the core 20 with the membrane 14.

In other embodiments, moving the core 20 relative to the housing 12 may include engaging the membrane 14 with the housing 12 (FIG. 21). In addition, moving the core 20 may include engaging the membrane 14 with a poke 24 on the housing 12.

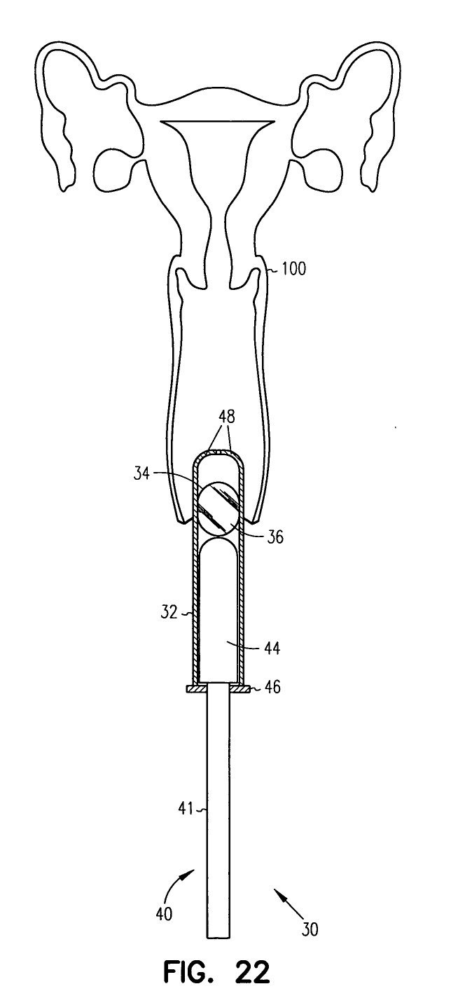

Another example method of supplying a material to a vagina will now be described with reference to FIGS. 22-24. As shown in FIG. 22, the method includes positioning a disposable device 30 near a vagina 100 (e.g., by inserting the disposable device 30 into the vagina 100). The disposable device 30 includes a housing 32 having a first end 46 and a second end 47, and a core 40 that is positioned within the housing 32. The disposable device 30 further includes a membrane 34 and a liquid 36 that is at least partially enclosed by the membrane 34.

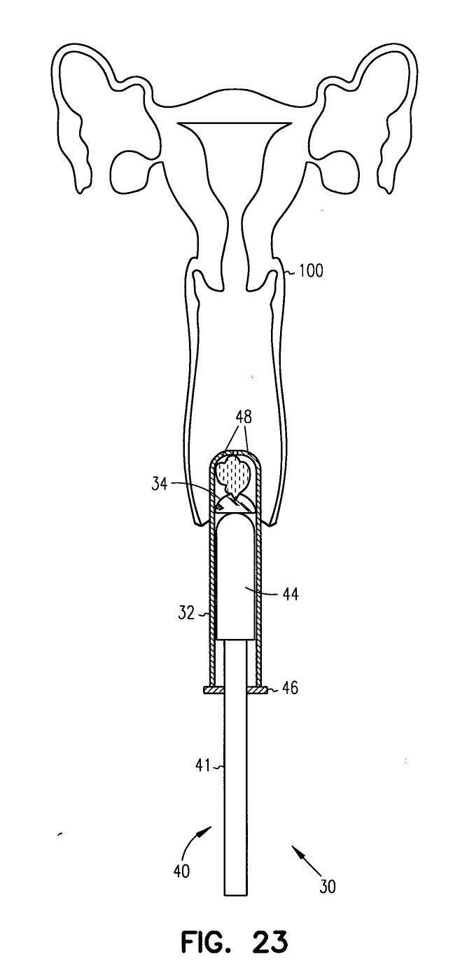

As shown in FIG. 23, the method further includes moving the core 40 relative to the housing 32 to open the membrane 34. It should be noted that moving the core 40 relative to the housing 32 to open the membrane 34 may include rupturing the membrane 34. In the example embodiments illustrated in FIGS. 22-24, the core 40 includes a handle 41 and a swab 44 that is secured to the handle 41 such that rupturing the membrane 34 may include rupturing the membrane 34 with the swab 44. In other embodiments, rupturing the membrane 34 may include rupturing the membrane 34 with the handle 41 (see, e.g., device 30 in FIGS. 9-11).

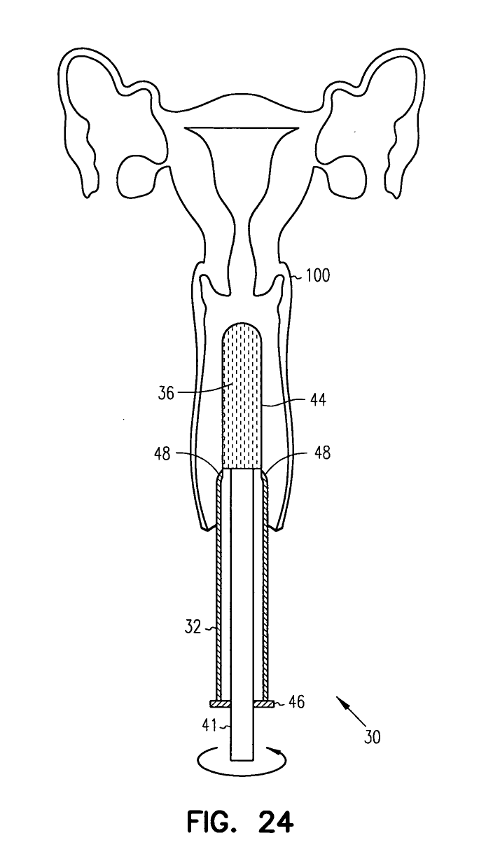

As shown in FIG. 24, the method may further include grasping the handle 41 of the core 40 and rotating the handle 41 to (i) clean inside the vagina 100 with the swab 44; and/or (ii) apply a material (e.g., a lubricant) to the vagina 100 with the swab 44. In some embodiments, the housing 32 includes a first end 46 and a second end 47 such that moving the core 40 relative to the housing 32 may include moving the core 40 between a first position (FIG. 22) where the swab 44 is at least partially within the housing 32 and a second position (FIG. 24) where the swab 44 is extended through the second end 47 of the housing 32.

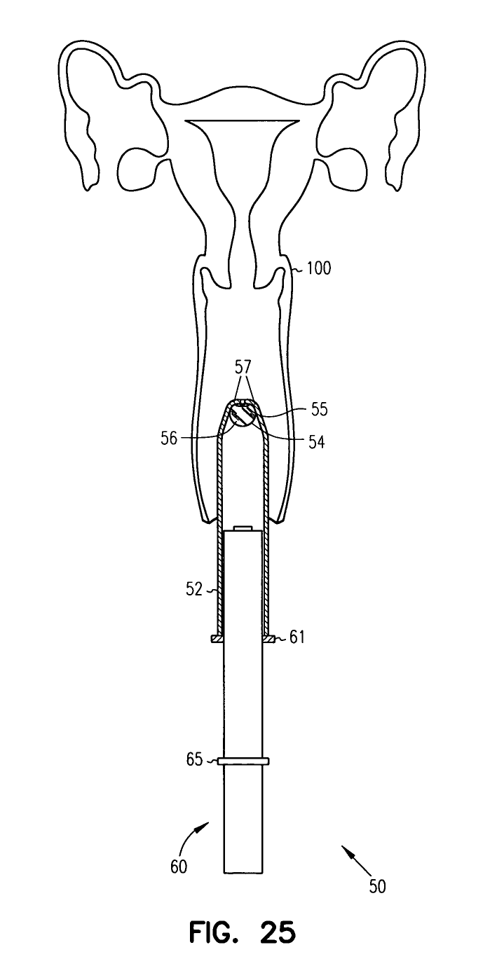

Another example method of supplying a material to a vagina will now be described with reference to FIGS. 25-27. The method includes positioning a disposable device 50 near a vagina 100. As an example, FIG. 25 shows inserting the disposable device 50 into the vagina 100. The disposable device 50 includes a housing 52 having a first end 61 and a second end 62, and a core 60 that is positioned within the housing 52. The disposable device 50 further includes a membrane 54 and a material (e.g., liquid 56) that is enclosed by the membrane 54. The membrane 54 includes an elastic member 55 that seals the liquid 56 inside the membrane 54.

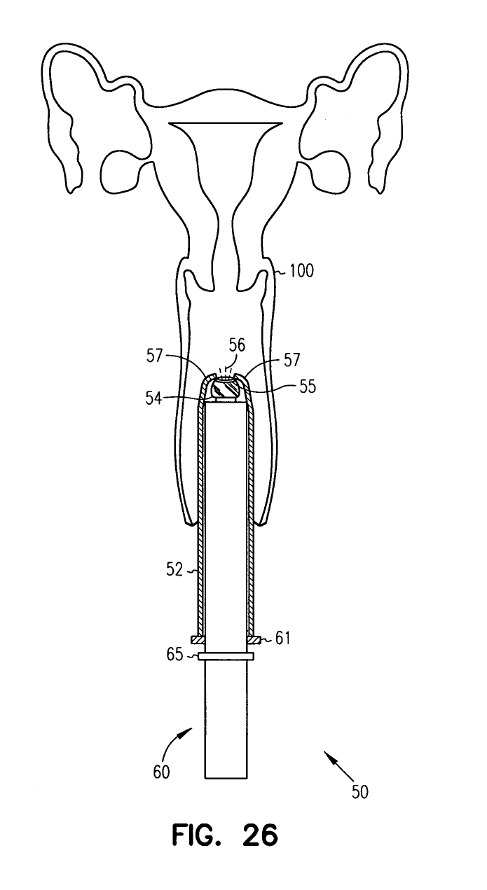

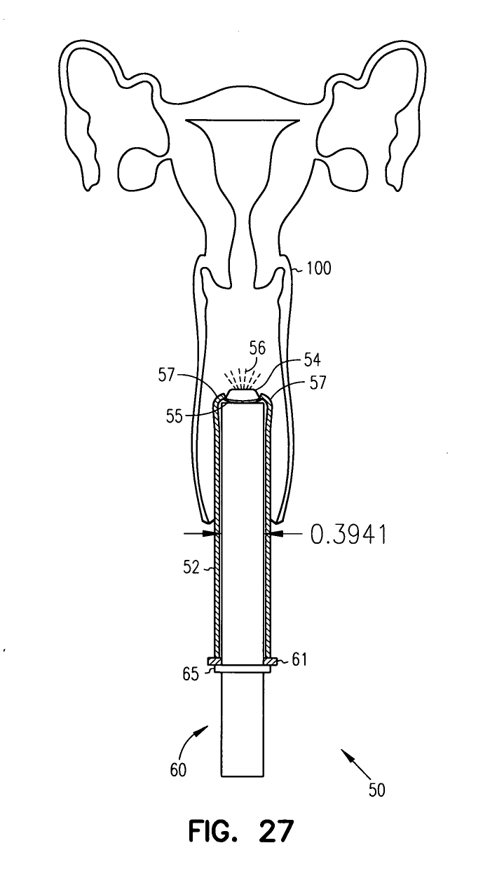

As shown in FIG. 26, the method further includes moving the core 60 relative to the housing 52 to open the membrane 54. In some embodiments, the second end 62 of the housing 52 includes resilient members 57 that close the second end 62 of the housing 52. The tips of the resilient members 57 engage the elastic member 55 of the membrane 54 such that moving the core 60 includes forcing apart the resilient members 57 to open the elastic member 55.

FIG. 27 shows that moving the core 60 relative to the housing 52 may further include forcing the membrane 54 through the elastic member 55 to dispense the liquid 56 within the vagina. It should be noted that other materials besides liquids may be stored within the disposable device 50. Depending on the type of material that is stored within the disposable device 50, the membrane 54 may need to be forced through the elastic member 55 in order to more thoroughly dispense the material within the vagina 100.

The method may further include engaging a stop 65 on the core 60 with the housing 52 such that the core 60 is prevented from further movement into the housing 52 (FIG. 24). The stop 65 may be positioned on the core 60 such that the stop 65 prevents the core 60 from moving far enough into housing 52 to dislodge the membrane 54 from the housing 52.

Any of the disposable devices shown and/or described herein may used in any of the methods of supplying a material to a vagina described herein. While the invention has been described in detail with respect to specific embodiments, it will be appreciated that there are variations of, and equivalents to these embodiments. Accordingly, the scope of the present invention should be determined by the appended claims and any equivalents thereto.

What is claimed is:

1. A disposable device for supplying a material to a vagina, the disposable device comprising:
a housing;
a membrane;
a material at least partially enclosed by the membrane; and
a core positioned within the housing, the core being movable within the housing to open the membrane.

2. The disposable device of claim 1 wherein the material is a liquid and the membrane is ruptured.

3. The disposable device of claim 2 wherein the core includes a handle and a swab that is secured to the handle.

4. The disposable device of claim 3 wherein the membrane may be ruptured such that the swab absorbs the liquid.

5. The disposable device of claim 3 wherein at least part of the handle is outside the housing.

6. The disposable device of claim 3 wherein the handle is embedded in the swab.

7. The disposable device of claim 3 wherein the housing includes a first end and a second end, and the core is movable between a first position where the swab is at least partially within the housing and a second position where the swab is extended through the second end of the housing.

8. The disposable device of claim 7 wherein the swab is positioned entirely within the housing when the core is in the first position.

9. The disposable device of claim 8 wherein the swab is positioned entirely outside the housing when the core is in the second position.

10. The disposable device of claim 8 wherein the second end of the housing includes resilient members that at least partially close the second end of the housing, the resilient members being forced apart by the swab as the core moves from the first position to the second position.

11. The disposable device of claim 3 wherein the housing includes a first end and a second end, the membrane being positioned between the swab and the second end of the housing before the membrane is ruptured.

12. The disposable device of claim 3 wherein the housing includes a first end and a second end, the membrane being positioned between the swab and the first end of the housing before the membrane is ruptured.

13. The disposable device of claim 2 wherein the membrane is attached to the housing before being ruptured.

14. The disposable device of claim 13 wherein the membrane and the housing enclose the liquid.

15. The disposable device of claim 2 wherein the membrane is attached to the core before being ruptured.

16. The disposable device of claim 15 wherein the membrane and the core enclose the liquid.

17. The disposable device of claim 2 wherein the liquid is enclosed by only the membrane.

18. The disposable device of claim 2 wherein the membrane is formed of plastic.

19. The disposable device of claim 2 wherein the core includes a poke that ruptures the membrane.

20. The disposable device of claim 2 wherein the housing includes a poke that ruptures the membrane.

21. The disposable device of claim 2 wherein the housing includes a first end and a second end and the disposable device further comprises a swab secured to the second end of the housing the membrane being within the swab such that the core ruptures the membrane within the swab as the core moves relative the housing.

22. The disposable device of claim 21 wherein the core includes a stop that engages the housing to prevent further movement of the core into the housing.

23. The disposable device of claim 22 wherein the stop on the core prevents the core from damaging the swab.

24. The disposable device of claim 1 wherein the housing includes a first end and a second end and the membrane includes an elastic member that seals the material inside the membrane, the second end of the housing including resilient members that close the second end of the housing, the resilient members engaging the elastic member of the membrane such that the elastic member opens as the core moves within the housing to force the resilient members apart.

25. The disposable device of claim 24 wherein each of the resilient members include tips that engage the elastic member of the membrane.

26. The disposable device of claim 24 wherein the material is a liquid.

27. The disposable device of claim 24 wherein the material is a solid.

28. The disposable device of claim 24 wherein the elastic member is formed of spandex.

29. The disposable device of claim 24 wherein the core forces the membrane through the elastic band to dispense the material from the membrane to the vagina.

30. The disposable device of claim 24 wherein the core includes a stop that engages the housing to prevent further movement of the core into the housing.

31. A method of supplying a material to a vagina, the method comprising:
positioning a disposable device near a vagina, the disposable device including a housing, a membrane, a material at least partially enclosed by the membrane and a core that is positioned within the housing; and
moving the core relative to the housing to open the membrane.

32. The method of claim 31 wherein positioning a disposable device near a vagina includes inserting a disposable device into a vagina.

33. The method of claim 31 wherein the material is a liquid such that moving the core relative to the housing to open the membrane includes rupturing the membrane.

34. The method of claim 33 wherein the core includes a handle and a swab that is secured to the handle such that rupturing the membrane includes rupturing the membrane with the swab.

35. The method of claim 33 wherein the core includes a handle and a swab that is secured to the handle such that rupturing the membrane includes rupturing the membrane with the handle.

36. The method of claim 33 wherein the core includes a handle and a swab such that moving the core relative to the housing includes grasping the handle of the core.

37. The method of claim 36 further comprising rotating the handle of the core to engage the swab with the vagina.

38. The method of claim 37 wherein rotating the handle of the core to engage the swab with the vagina includes rotating the handle to apply the material to the vagina.

39. The method of claim 37 wherein rotating the handle of the core to engage the swab with the vagina includes rotating the handle to clean the vagina.

40. The method of claim 36 wherein the housing includes a first end and a second end such that moving the core relative to the housing includes moving the core between a first position where the swab is at least partially within the housing and a second position where the swab is extended through the second end of the housing.

41. The method of claim 33 wherein moving the core relative to the housing includes engaging the core with the membrane.

42. The method of claim 41 wherein moving the core includes engaging a poke on the core with the membrane.

43. The method of claim 33 wherein moving the core relative to the housing includes engaging the membrane with the housing.

44. The method of claim 43 wherein moving the core includes engaging the membrane with a poke on the housing.

45. The method of claim 31 wherein the housing includes a first end and a second end and the membrane includes an elastic member that seals the material inside the membrane, the second end of the housing including resilient members that close the second end of the housing, the resilient members engaging the elastic member of the membrane such that moving the core includes forcing apart the resilient members to open the elastic member.

46. The method of claim 45 wherein moving the core relative to the housing includes forcing the membrane through the elastic member to dispense the material within the vagina.

47. The method of claim 46 wherein forcing the membrane through the elastic member to dispense the material within the vagina includes dispensing a powder within the vagina.

48. The method of claim 46 wherein forcing the membrane through the elastic member to dispense the material within the vagina includes dispensing a liquid within the vagina.

49. The method of claim 45 further comprising engaging a stop on the core with the housing such that the core is prevented from further movement into the housing.
